(19) United States
(12) Patent Application Publication
(10) Pub. No.: US 2005/0119592 A1
(43) Pub. Date: Jun. 2, 2005

(54) Device for analyzing the support reaction of the lower extremities for diagnosis of the state of the human spinal column

(76) Inventors: Zuzana Frank, Leipheim (DE); Marina Tretjakova, Moskau (RU)

Correspondence Address: WILLIAM COLLARD;COLLARD & ROE, P.C., 1077 NORTHERN BOULEVARD, ROSLYN, NY, 11576, US

(21) Appl. No.: 10/491,454

(22) PCT Filed: Sep. 30, 2002
(86) PCT No.: PCT/EP02/10933

(30) Foreign Application Priority Data
Oct. 1, 2001 (DE) 101 48 529.8

(51) Int. Cl. A61B 5/103; A61B 5/117

(52) U.S. Cl. 600/594; 600/592; 600/595

(57) ABSTRACT

The invention relates to a device for analyzing the support reaction of the lower extremities for diagnosis of the state of the human spinal column, having a right support stage (1) and a left support stage (2), which are each at least adjustable in height and mounted to rotate about two horizontal axes and have a transparent tread (6). An electronic unit (15) for optical detection of the plantar surface of the human foot assigned to the support stage (1, 2), in each instance, is arranged underneath each tread (6). Each unit (15) is connected to a computer system (7).

The invention relates to a device for analyzing the support reaction of the lower extremities for diagnosis of the state of the human spinal column, having a right support stage and a left support stage, which are each at least adjustable in height and mounted to rotate about two horizontal axes and have a transparent tread.

The human spinal column as the central supporting element of the skeleton plays a significant role in determining the state of health of a person, whereby incorrect positions of individual vertebrae and the resulting state of the spinal column, which deviates from the natural position and curvature, can detrimentally affect the health of a person in many different ways. Some disease profiles, such as attacks of dizziness, appear to have nothing to do with the spinal column, but can be caused by the vertebrae if, for example, the disks positioned between the vertebrae press on the nerves of the spinal cord. In addition to the correct alignment of adjacent vertebrae relative to one another, the spinal column must also be considered as a whole, in order to assess its scoliosis. However, the spinal column can only exercise its supporting function in interaction with the lower extremities, which must bear the entire load in the normal, natural posture and location of a person. Incorrect positions of the spinal column can be caused by a different length of the two legs of a person, who attempts to compensate the difference in length of his/her legs by means of a corresponding posture of his/her upper body. Vice versa, however, an apparent difference in length of the two legs can also result from a slanted position of the pelvis, caused by the spinal column. In the end result, a detailed and careful analysis of the chain of force transfer from the lower extremities to the spinal column, with the joints involved in this, is required, in order to be able to balance out incorrect positions of the spinal column and achieve its natural position.

For a diagnosis of the state of the spinal column, it has already been proposed to balance out different leg lengths by providing support stages having different height positions. However, this has proven to be insufficient and has led to unsatisfactory results, since the other biomechanical consequences of the different height position of the support stages were still left out of consideration, namely that lifting one leg is necessarily connected with a rotation of the foot that supports this leg, in the ankle, and this rotation could not be assessed, either qualitatively or quantitatively, until now. Devices that are solely designed for examining or exercising the ankle joint, such as that described in U.S. Pat. No. 6,162,189, are also not able to do this.

The invention is therefore based on the task of configuring a device of the type stated initially, in such a manner that the support reaction of the lower extremities can be assessed and characterized.

This task is accomplished, in the case of a device of the type stated initially, in that a first electronic unit for optical detection of the plantar surface of the human foot assigned to the support stage, in each instance, is arranged underneath each tread, and that each unit is connected to a computer system.

This device, for the first time, results in the possibility of determining not only the height position of the right and the left support stage, i.e. their difference, but also the angle of slant of the foot in its longitudinal direction, and the direction perpendicular to that, specifically in a predetermined and reproducible state, for which the contact of the plantar surface of the foot with the tread is characteristic, particularly by means of complete surface contact, in other words with a maximal contact surface that can be achieved, which is measured by means of the unit and recorded and assessed in the computer system, particularly for the purpose of determining changes in the case of modification of the posture.

A particularly preferred embodiment of the invention is characterized in that each support stage is mounted to rotate on a drive stage, about three axes that stand perpendicular to one another, and can be adjusted in linear manner, in the direction of these axes. This embodiment, with an expanded adjustment possibility of the support states in a total of six degrees of freedom, makes an adaptation to the existing anatomic conditions of the person possible, particularly an influence on his/her stance width, which should be selected as a function of the size of the person and, in particular, his/her shoulder width.

It is furthermore advantageous if the support stages are jointly arranged in a platform on a rotation module, which is mounted to rotate about a vertical axis arranged to run symmetrically between the two support stages. This embodiment also allows a rotation of the lower extremities about the longitudinal axis of the body, so that another parameter can be varied and determined to define the state of the spinal column, in other words the mobility in the hips or a rotation of the upper body is taken into consideration.

It has also proven to be practical that in order to adjust the support stages as well as the rotation module, actuators that can be activated by means of force are provided, which are connected with the computer system in order to be monitored and controlled. Such actuators for adjusting the support stage relative to the drive stage, in six degrees of freedom, are known from U.S. Pat. No. 5,987,726, and therefore do not have to be explained in greater detail here. The only thing that needs to be emphasized is the advantage of tying these force-activated actuators in with the computer system, with regard to an automated sequence in the examination of the status of the spinal column.

It has also proven to be practical if the optical unit is formed by a scanner or a digital camera or a video camera, which are commercially available in many different embodiments, and allow integration into the computer system, without problems.

In order to obtain informative examination results, it is advantageous if each support stage has a scale connected with the computer system assigned to it, since in this way, there is the possibility of determining whether or not a uniform load is placed on both support stages, independent of the person's sense of equilibrium or ability to cooperate.

It is furthermore advantageous if the computer system includes not only the monitor for the operator but also a data monitor for the person, since in this way, the person's ability and willingness to cooperate can be increased and, at the same time, direct feed-back to the person about his/her current posture and variations that must be made in order to achieve the desired standing position is possible. The data monitor is arranged on a column that is arranged in the middle in front of the two support stages, which is adjustable in height, in order to thereby be able to adapt the location of the data monitor to the size of the person and allow this person to observe the data monitor without turning his/her head at the cervical spinal column or other parts of the spinal column.

It is practical if the column has a safety railing assigned to it, which can be utilized in order to prevent the person from losing his/her equilibrium. In this embodiment, it is advantageous if the safety railing has a scale connected with the computer system assigned to it, so that it can be reliably determined, in the analysis of the status of the spinal column, that no noteworthy support of the person was provided by way of the safety railing, but rather his/her weight was resting almost completely on the two support stages.

In order to obtain a defined starting position for the start of the examinations, guide rails for a correct orientation of the person's feet are arranged on each tread, particularly in such a manner that the guide rails are provided for contact on the heel zone and the inside zone of each foot, and predetermine an angle of the longitudinal foot axes from 12° to 20°, preferably 16°.

In order to reduce the amount of time required to perform the examination, and be able to achieve the status of a uniform load on the two support stages as quickly as possible, a digital display for displaying the relative load on the support stages, particularly for displaying that the load on them is uniform, is provided; it is practical if this display is assigned to the data monitor, and thereby allows the person to adjust the weight distribution to the requirements himself/herself.

For recording the examination and the automated assessment, a second electronic unit for optical detection of the state of the spinal column is provided, which also allows a reproduction of the image of the spinal column that was taken on the data monitor, if it is connected to the computer system.

Each tread has a plurality of pressure sensors for detecting the pressure in the contact surface between the foot and the tread, with local resolution, which sensors supplement and support the information obtained by the first electronic unit for optical detection.

The invention will be explained in greater detail in the following, using exemplary embodiments shown in the drawing; the figures show:

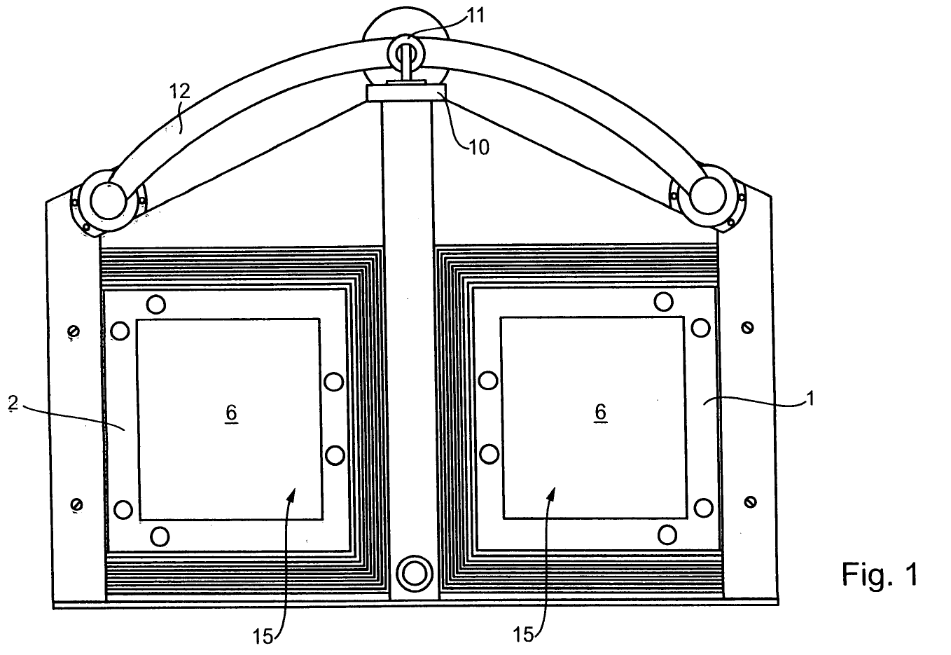

FIG. 1 a top view of a first exemplary embodiment of the device according to the invention;

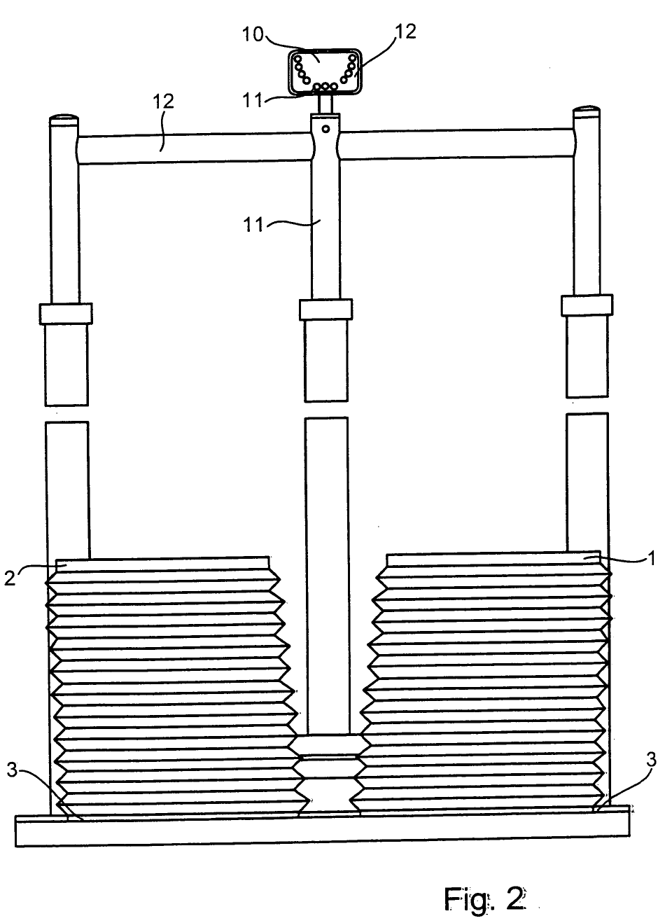

FIG. 2 a front view of the device from FIG. 1;

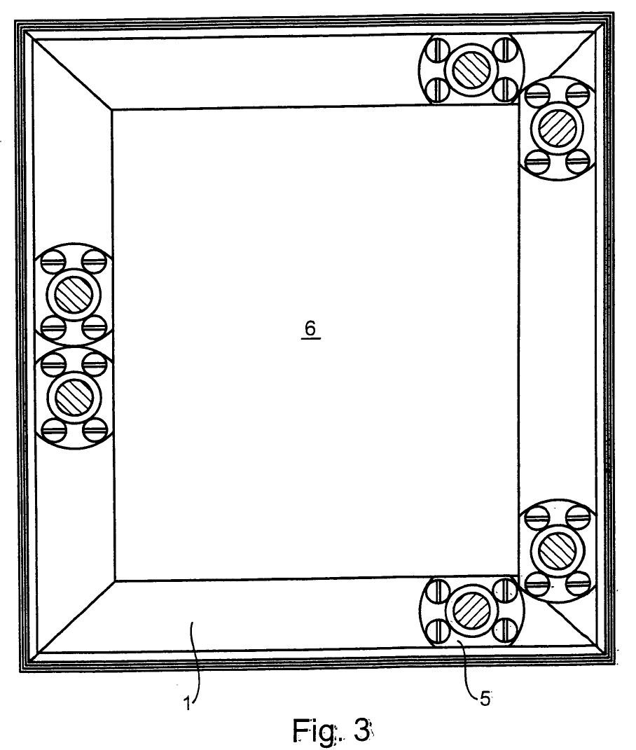

FIG. 3 a top view of the support stage;

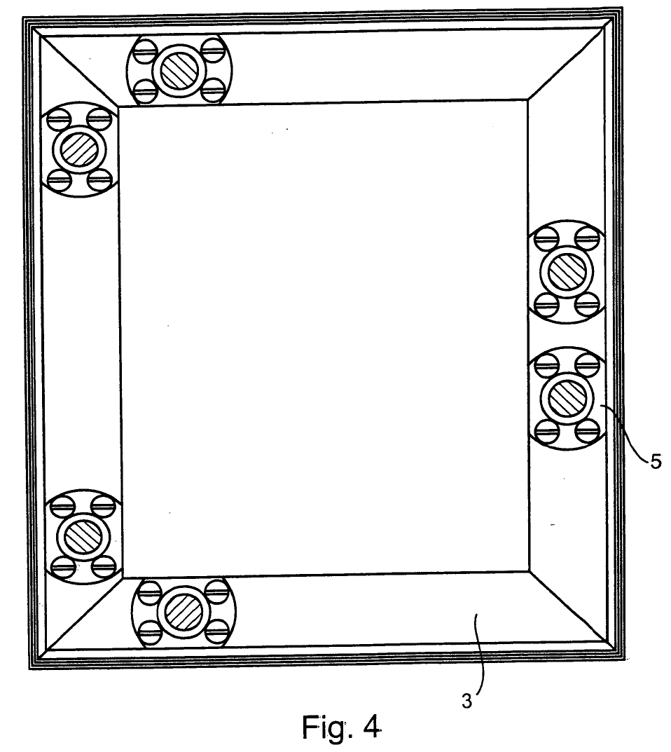

FIG. 4 a top view of the drive stage;

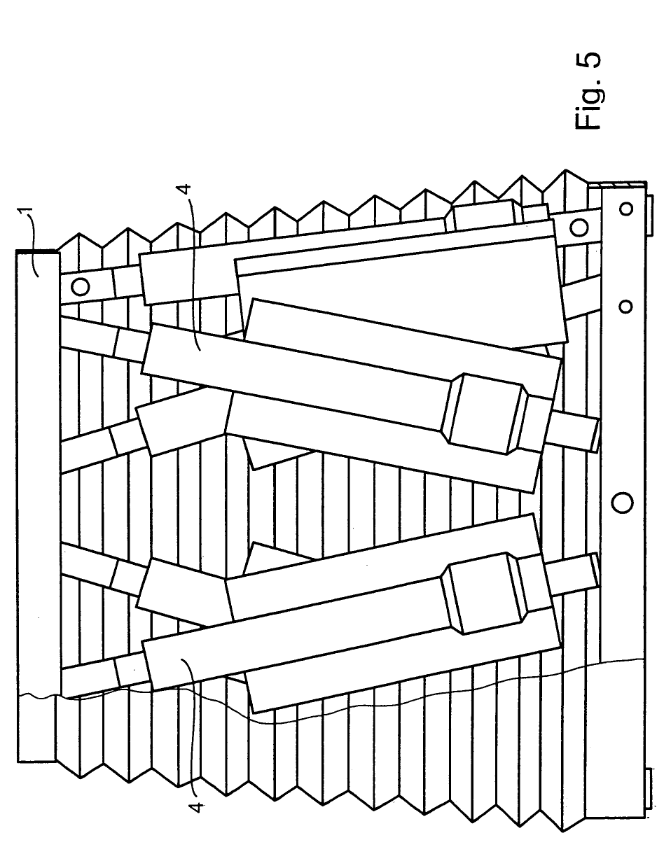

FIG. 5 a side view of the actuators arranged between the drive stage and the support stage,

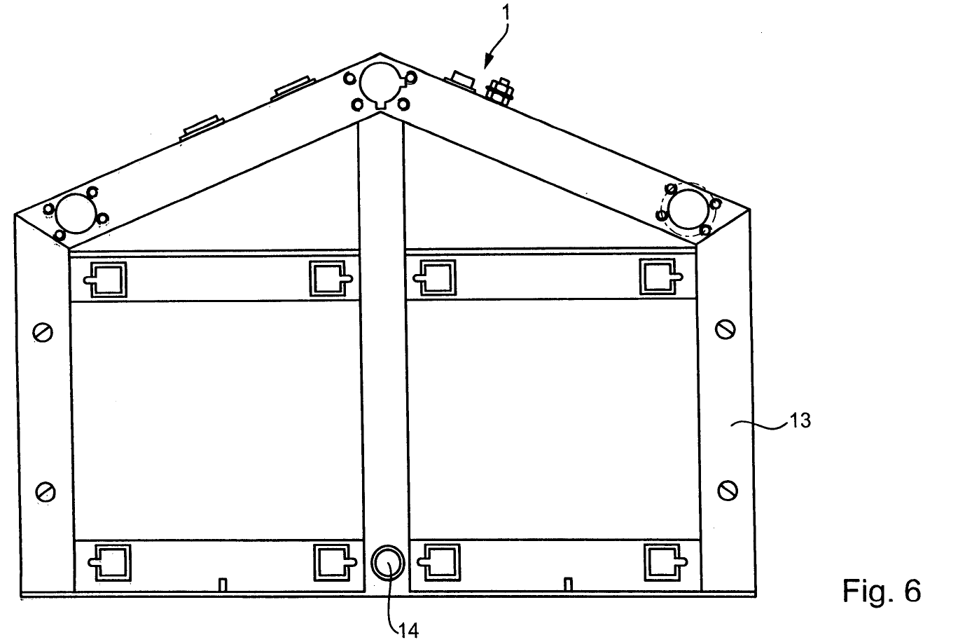

FIG. 6 a top view of the rotation module, and

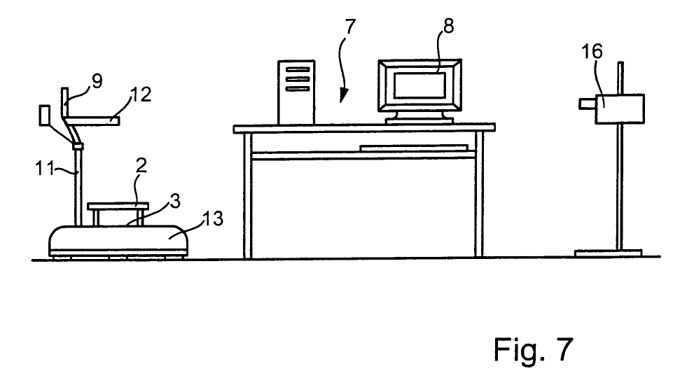

FIG. 7 a schematic representation of a second embodiment.

FIG. 1 shows a device that serves to analyze the support reaction of the lower extremities of a person, for diagnosis of the state of his/her spinal column. This device has a right support stage 1 as well as a left support stage 2, which are each mounted on a drive stage 3 and have six degrees of freedom, namely they can be rotated about three axes that stand perpendicular to one another, and are adjustable, in linear manner, in the direction of these axes. To adjust the support stages 1, 2, actuators 4 that can be activated by force are provided, which are visibly shown in FIG. 5, while only their connections 5 to the support stage 1 and the drive stage 3 can be seen in FIG. 1 as well as in FIGS. 3 and 4.

Each support stage 1, 2, has a transparent tread 6, underneath which an electronic unit 15 for optical detection of the plantar surface of the human foot assigned to the support stage 1, 2, in each instance, is arranged. Both a scanner and a digital camera or a video camera are suitable for implementing the optical unit, whereby the term “transparent” relates to the part of the spectrum detected by the optical unit, which can correspond to the IR range, for example, when using a heat image camera.

The device is furthermore supplemented in that each support stage 1, 2 has a scale assigned to it, which can be used to determine the load on each support stage 1, 2.

Both the optical unit 15 and the actuators 4 and the scales are connected with a computer system 7, which has not only the monitor 8 for the operator but also a data monitor 9 for the person, in order to allow direct feed-back about the effects of his/her actions (FIG. 7).

FIG. 2 shows an embodiment in which the device has a signal device 10 for displaying the relative load on the support stages 1, 2 assigned to it, whereby this signal device 10 is formed by a plurality of signal lamps 11, 12, in the exemplary embodiment shown, some of which (12) are assigned to one of the two support stages 1, 2 and light up if the load on this support stage 1, 2 is greater than the load on the other support stage 1, 2. Some of the signal lamps 11 are provided indicating that the load is uniform. The signal device 10 is arranged on a column 11 that is arranged in the middle in front of the support stages 1, 2 and is adjustable in height, which furthermore has a safety railing 12 assigned to it, which has another scale connected with the computer system 7.

The device has a rotation module 13 that is adjustable by means of actuators that are also connected with the computer system 7, in which the support stages 1, 2 are jointly arranged in a platform, whereby the rotation module 13 is mounted so as to rotate about a vertical axis 14 that runs symmetrically between the two support stages 1, 2.

The use of this device for analyzing the support reaction of the lower extremities takes place in such a manner that first, a person steps onto the support stage 1, 2, whereby he/she can grasp the safety railing 12 in order to prevent a loss of equilibrium. With regard to this starting position, an image of the plantar surface of the feet is taken by means of the first optical unit 15, and stored in the memory of the computer system 7, for a comparison. In interaction with the operator, or in automated manner, one or also both support stages 1, 2 can then be adjusted, in order to make a change, proceeding from the state of the spinal column that exists for the person, and in order to achieve a natural position of the spinal column. By way of imaging the plantar surface of both feet, the foot contact of the person with the tread 6 of the support stages 1, 2 is checked, in order to determine the parameters that characterize the spatial orientation of the support stages 1, 2 and thereby the support reaction of the lower extremities, if the load on the support stages 1, 2 continues to remain uniform, as checked by means of the scales, via the signaling device 10. By means of a second electronic unit 16 for optical detection of the state of the spinal column, for example the video camera shown in FIG. 7, the state of the spinal column can be detected, in order to then be able to make suggestions for therapy, with which the desired state of the spinal column can be permanently achieved by means of inserts, orthopedic shoes, or the like.

1. Device for analyzing the support reaction of the lower extremities for diagnosis of the state of the human spinal column, having a right support stage (1) and a left support stage (2), which are each at least adjustable in height and mounted to rotate about two horizontal axes and have a tread (6), characterized in that the treads (6) are transparent, that an electronic unit (15) for optical detection of the plantar surface of the human foot assigned to the support stage (1, 2), in each instance, is arranged underneath each tread (6), that each unit (15) is connected to a computer system (7), and that the support stages (1, 2) are jointly arranged in a platform, on a rotation module (13), which is mounted so as to rotate about a vertical axis (14) that runs symmetrically between the two support stages (1, 2).

2. Device according to claim 1, wherein each support stage (1, 2) is mounted on a drive stage (3), so as to rotate about three axes that stand perpendicular to one another, and can be adjusted, in linear manner, in the direction of these axes.

3. (canceled)

4. Device according to claim 1, wherein the support stages (1, 2) are jointly arranged in a platform, on a rotation module (13), which is mounted so as to rotate about a vertical axis (14) that runs symmetrically between the two support stages (1, 2).

5. Device according to claim 1, wherein the optical unit (15) is formed by a scanner or a digital camera or a video camera.

6. Device according to claim 1, wherein each support stage (1, 2) has a scale connected with the computer system (7) assigned to it.

7. Device according to claim 1, wherein the computer system (7) has not only a monitor (8) for the operator but also a data monitor (9) for the person.

8. Device according to claim 7, wherein the data monitor (9) is arranged on a column (11) that is arranged in the middle in front of the two support stages (1, 2), which is adjustable in height.

9. Device according to claim 8, wherein characterized iii that the column (11) has a safety railing (12) assigned to it.

10. Device according to claim 9, wherein safety railing (12) has a scale connected with the computer system (7) assigned to it.

11. Device according to claim 1, wherein guide rails for a correct orientation of the person's feet are arranged on each tread (6).

12. Device according to claim 11, wherein the guide rails are provided for contact on the heel zone and the inside zone of each foot, and predetermine an angle of the longitudinal foot axes from 12° to 20°, preferably 16°.

13. Device according to claim 6, wherein a signal device (10) for displaying the relative load on the support stages (1, 2), particularly for displaying that the load on them is uniform, is provided.

14. Device according to claim 12, wherein the signal device (10) is assigned to the data monitor (9).

15. Device according to claim 1, wherein a second electronic unit (16) for optical detection of the state of the human spinal column is provided.

16. Device according to claim 1 that each tread (6) has a plurality of pressure sensors assigned to it, for detecting the pressure in the contact surface between the foot and the tread (6), with local resolution.
